(19) United States
(12) Patent Application Publication
(10) Pub. No.: US 2019/0313895 A1
(43) Pub. Date: Oct. 17, 2019

(54) SYSTEM AND METHOD FOR AUTOMATIC ASSESSMENT OF DISEASE CONDITION USING OCT SCAN DATA

(71) Applicant: Tecumseh Vision, LLC, Pittsburgh, PA (US)

(72) Inventors: James Hayashi, Pittsburgh, PA (US); Ravi Starzl, Pittsburgh, PA (US); Hugo Angulo, Pittsburgh, PA (US); Abhishek Kar, Pittsburgh, PA (US); Ramesh Oswal, Pittsburgh, PA (US); Diego Penafiel, Pittsburgh, PA (US); Weidong Yaun, Pittsburgh, PA (US)

(21) Appl. No.: 16/462,360

(22) PCT Filed: Nov. 21, 2017
(86) PCT No.: PCT/US17/62747

§ 371 Date: May 20, 2019

Related U.S. Application Data
(60) Provisional application No. 62/424,832, filed on Nov. 21, 2016.
(60) Provisional application No. 62/254,681, filed on Nov. 12, 2015.

(51) Int. Cl. A61B 3/10; A61B 3/14; G06K 9/46; G06K 9/62; G06N 3/04; G06T 7/00

(52) U.S. Cl. CPC A61B 3/102; A61B 3/14; G06K 9/4628; G16H 30/40; G06N 3/0454; G06T 7/0012; G06K 9/6273

(57) ABSTRACT

Machine learning algorithms are applied to OCT scan image data of a patient's retina to assess various eye diseases of the patient, such as ARMD, glaucoma, and diabetic retinopathy. The classification modules for each tested-for disease or condition preferably comprises an ensemble of machine learning algorithms, preferably including both deep learning and traditional machine learning (non-deep learning) algorithms. The results of the analysis can be transmitted back to the facility of the caregiver that used the OCT scanner to scan the patient's retina while the patient is still present at the caregiver's facility for an appointment.

### PRIORITY CLAIM

The present application claims priority to U.S. provisional applications Ser. No. 62/424,832, filed Nov. 21, 2016 and Ser. No. 62/524,681, filed Jun. 26, 2017, both of which are incorporated herein by reference in their entirety.

### BACKGROUND

Age Related Macular Degeneration (ARMD) is the leading cause of blindness in the United States. ARMD is commonly thought to exist in two forms—“dry” and “wet.” The wet form often results when a choroidal neovascular membrane (CNVM) has growth beneath the retina. A CNVM often results in sudden, severe vision loss which, if left untreated, is permanent. In the Age Related Eye Disease Studies (or AREDS and AREDS2), vitamin therapy has been shown to treat dry macular degeneration somewhat effectively. Vitamin therapy is recommended for people who have moderate and/or severe cases of the disease, although the benefit is minimal for people with mild cases. The current recommendation is that people with dry macular degeneration self monitor using an Amsler grid, along with an examination of their retina every six months. Most patients stay dry and only 10-15% of macular degeneration patients develop a case of wet ARMD. Once they get wet ARMD, the most effective way to treat them is with injections inside the eye with anti vascular endothelial growth factor (or anti-VEGF) injections. The frequency of the need of these injections is highly variable and a point of considerable controversy. Currently, Medicare spends over 3 billions dollars on these injections and most of this cost may be unnecessary.

Another eye disease is diabetic retinopathy, which is the leading cause of visual disability among working age adults. An estimated 25 million Americans have been diagnosed, which is a small proportion to the complete number. Numerous clinical trials have shown that early intervention in diabetic eye disease, with ophthalmic lasers and anti vascular endothelial growth factor agents, has a profound beneficial effect on the natural progression of the disease. Current therapies have shown to be about 90% effective in preventing severe visual loss (visual acuity <5/200). The American Academy of Ophthalmology and the American Diabetes Association recommend routine screening protocols. However, despite the proven benefit of early detection, annual exams are only followed approximately 50% of the time, and annual exam rates may be as low as 30% in high risk groups. Although the treatment of diabetes has led to a decrease in diabetic eye disease prevalence, the overall increase in the prevalence of diabetes has meant that the eye disease burden has not lessened. Because of modernization and the spread of Western dietary practices, diabetes, has unfortunately become a worldwide epidemic.

Another major cause of irreversible blindness is glaucoma, in particular, primary open angle glaucoma (POAG). POAG affects over 2 million Americans and the numbers are expected to increase as the population ages. There are over 8 million people blind from glaucoma worldwide. Primary open angle glaucoma is an ideal disease for screening because it has a reasonably high prevalence in the population, is asymptomatic early in the course of disease, and can slow or even eliminate visual field loss if detected and treated early. Screening for glaucoma is problematic though, because measuring the intraocular pressure has been shown to be very ineffective as a screening measure.

### SUMMARY

Therefore, in one general aspect, the present invention is directed to systems and methods for applying machine learning to Optical Coherence Tomography (OCT) scan data of a patient's retina. It is capable of detecting the presence and/or state of disease conditions in the patient, particularly eye-related diseases, such as ARMD, glaucoma (e.g., POAG), and/or diabetic retinopathy. Embodiments of the present invention could also be used to detect other disease conditions from OCT retina scan image data, such cardiovascular, Alzheimer's and/or Parkinson's disease.

By using OCT scan data according to the present invention, early detection for the various disease conditions can be improved. Moreover, currently most OCT machines are located at an eye doctor's office. Enhancing the functionality of OCT machines to detect for other diseases, results in the economic incentive to include OCT machines at the offices of primary care providers. Screening for various eye disease conditions can move from an eye specialist's setting to a primary care doctor's office—all while the patient remains at the primary care doctor's office for a visit. Moreover, such automated assessments can help cover for the shortage in retinal specialists that diagnose patients. Additionally, the automated assessments can improve patient convenience (e.g., by having the assessment performed while the patient is at his/her primary care provider's office for an appointment) and compliance (e.g., by better identifying when follow-up treatment is needed).

These and other benefits realizable through various embodiments of the present invention will be apparent from the description that follows.

### FIGURES

Various embodiments of the present invention are described herein by way of example in conjunction with the following figures, wherein:

FIG. 1A is a diagram of an apparatus according to various embodiments of the present invention;

FIG. 1B is a diagram of an OCT scanner according to various embodiments of the present invention;

FIG. 1C is an example of OCT scan image of the retina;

FIG. 1D is a flowchart depicting a process flow of the host computer system of the apparatus of FIG. 1A according to various embodiments of the present invention;

FIG. 2A depicts a graphical representation of LeNet;

FIG. 2B depicts a graphical representation of AlexNet;

FIG. 3 depicts a method for developing and training the statistical models of the apparatus of FIG. 1A according to various embodiments of the present invention; and

FIG. 4 illustrates an ensemble of statistical methods.

### DESCRIPTION

Ocular Coherence Tomography (OCT) is an established medical imaging technology that uses light and analysis of the scattering of the light by the biological tissue to produce high resolution images on the micrometer scale. One can think the images in terms of low powered microscopic slides. The application of statistical modeling techniques to OCT images from human retinas in the above use cases, can detect the presence and/or state of eye disease conditions in patients. The present invention, in one embodiment, can effectively leverage traditional machine learning and deep learning methodologies for the detection of diseases, such as ARMD, glaucoma and/or diabetic retinopathy. Moreover, embodiments of the present invention can be used to accurately address many of the clinical questions of these conditions, often in a manner not requiring a highly trained specialist, such as a retina doctor, to read the images. For example, questions that can be addressed by the system of the present invention can include: does the patient have ARMD, for example. If so, will the patient benefit from vitamin therapy? Does the ARMD patient now have wet ARMD? If the patient has wet ARMD, will they require frequent or less frequent injections? And if the patient has been treated and responded to anti-VEGF injections, has there been a recurrence of the CNVM? (hereinafter “the follow-up questions”). Similarly, if the patient is diagnosed with glaucoma, the follow-up questions can include whether the patient's glaucoma is severe, such that it needs to be treated soon, or not so severe such that treatment can be delayed.

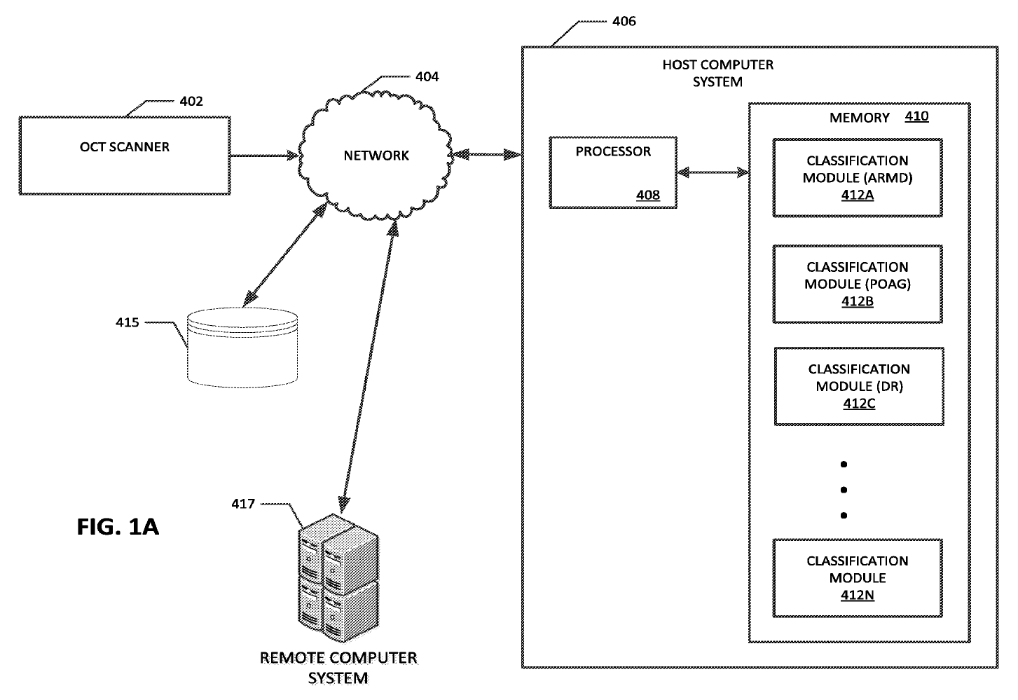

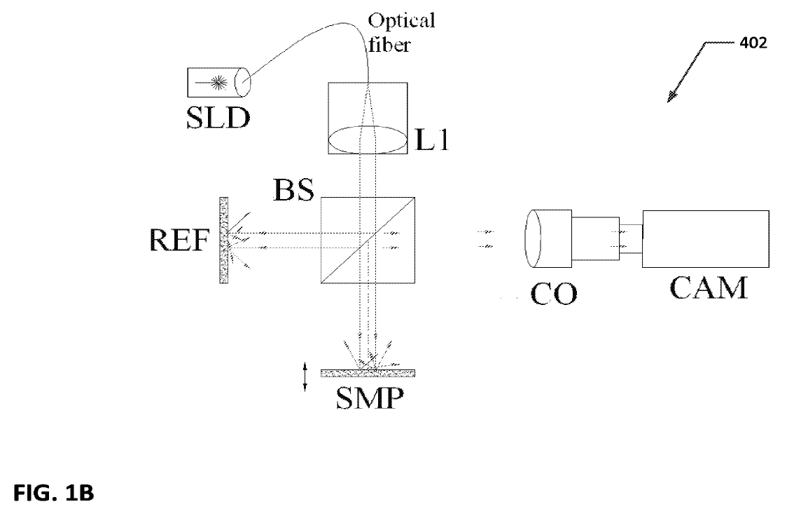

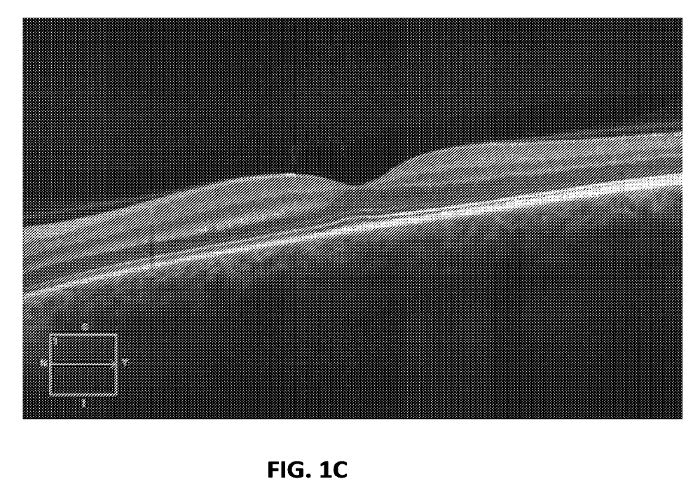

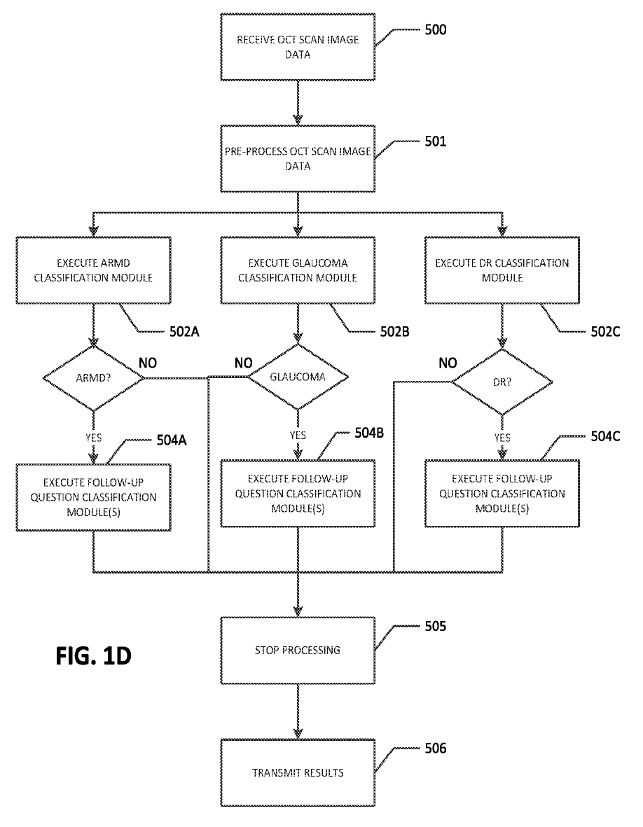

FIG. 1A is a diagram of a system 400 according to various embodiments of the present invention. The system 400 comprises an OCT scanner 402 and a host computer system 406. The OCT scanner 402 is a medical imaging device that uses light (usually infrared light) to capture micrometer-resolution, three-dimensional images from within optical scattering media (e.g., biological tissue), such as a human's retina. The OCT scanner 402 may include an interferometer (e.g., a Michelson type) with a low coherence, broad bandwidth light source, such as a super-luminescent diode (SLD) or laser. FIG. 1B is a diagram of an OCT scanner according to various embodiments of the present invention. Light from the light source, e.g., the SLD, is projected through a convex lens L1, and then split into two beams by a beam-splitter (BS). One beam is directed to a reference (REF) and the other beam is directed to the sample (SMP), e.g., the patient's retina. The reflected light from the sample and reference paths are recombined. A light detector, such as a camera (CAM in FIG. 1B) or photodetector, collects the images data for digital processing. FIG. 1C provides an example of an OCT scan of a retina, in this case of a relatively healthy macula portion of the retina.

Scan retina image data (or other body part, depending on what the types of diseases being diagnosed) collected by the OCT scanner 402r from a patient (or patients), may be transmitted to the host computer system 406 via a data network 404, such as the Internet, a WAN or LAN, etc. In addition or alternatively, the OCT scanner 402 could upload the scan image data to a database 415, such as a network or cloud-based database, and the host computer system 406 could then download the scan image data from the database 415 for processing.

As described below, the host computer system 406 statistically analyzes the scan data for the patient (or patients), to determine a likelihood that the patient has (or the patients have) the tested-for disease(s), e.g., ARMD, glaucoma, or diabetic retinopathy in one embodiment. If any of those eyes diseases are identified, it analyzes additional features of the eye-disease (e.g., the “follow-up questions” described above). That is, the host computer system 406 may employ machine learning techniques to classify the patients as having an eye disease based on the patient's OCT scan image data and to address follow-up questions (each being a classification task), using traditional machine learning and/or deep learning techniques. The host computer system 406 may employ an ensemble of traditional machine learning and/or deep learning algorithms to make the classifications as described below. The host computer system 406 could be co-located with the OCT scanner 402 or remote from the OCT scanner 402. For embodiments where they are co-located, the OCT scanner 402 and the host computer system 406 may be in communication via a wired communication link (e.g., Ethernet) or a wireless communication link (e.g., WiFi). For embodiments where the OCT scanner 402 and the host computer system 406 are remote, they can be in communication via the electronic data network 404. As shown in FIG. 1A, the host computer system 406 may comprise one or a number of networked computer devices, such as PCs, laptops, servers, etc. Where the host computer system 406 comprises multiple computer devices, they may be co-located or distributed across a network. In that connection, the host computer system 406 comprises one or more processor(s) 408 and one or more associated memory units 410 (only one of each is shown in FIG. 1A for simplicity) that store software for execution by the processor(s) 408. The memory unit(s) 410 may comprise primary and secondary computer memory. The primary memory may be directly accessible by the processor(s) 408. The processor(s) may continuously read instructions (e.g., software) stored in the primary memory and execute the instructions as required. The primary memory can comprise RAM, ROM, processor registers and processor cache memory. The secondary memory can comprise storage devices that are not directly accessible by the processors, such as HDDs, SSDs, flash optical data storage units, magnetic tape memory, etc. Any data actively operated on by the processor(s) 408 may be stored in the primary and/or secondary memory.

The processor(s) 408 preferably comprises multiple processing cores, such as multiple CPU or GPU cores. GPU cores operate in parallel and, hence, can typically process data more efficiently that a collection of CPU cores, but all the cores execute the same code at one time. GPUs are particularly better suited for deep neural networks, as described below.

According to various embodiments, as shown in FIG. 1A, the memory unit(s) 410 comprises one or more pre-trained classification modules 412A to 412N. The classification modules 412A-N store computer instructions, e.g., software, that is executed by the processor(s) 408 in order to perform the statistical analysis on the OCT scan data received from the OCT scanner 402. Each classification module 412 can be “tuned” or “trained” to a particular classification question, e.g., whether the patient has a particular disease and/or follow-up questions. For example, as shown in the example of FIG. 1A, a first classification module 412A can assess whether the patient has ARMD, a second classification module 412B can assess whether the patient has glaucoma (POAG), a third classification module 412C can assess whether the patient has diabetic retinopathy (DR), and other classification modules 412D-N can address relevant follow-up classifications.

Once trained, the classification modules 412A-N (which can each comprise an ensemble of traditional machine learning and/or deep learning algorithms) can make their respective classifications for a patient based on the patient's OCT scans. Accordingly, in (post-training) operation, the host computer system 406 receives the OCT scan data from the OCT scanner 402 for a patient, and then the processor(s) 408 executes the software for the classification modules 412A-N as needed to make their respective determinations, or classifications. To illustrate, the modules 412A-N can determine whether the patient has ARMD, and/or glaucoma, and/or diabetic retinopathy, etc. The determination by the host computer system 406 can include a probability based on its statistical analysis that the patient has tested-for condition or a binary output (yes or no). If probability exceeds some threshold (e.g., 50%) or if the condition result is yes in a binary determination, the classification modules can be executed to make their respective classifications for follow-up questions as needed. For example, if the ARMD classification module 412A determines that the patient likely has ARMD, the classification modules specific to the ARMD diagnosis can be executed (e.g., in case of wet ARMD—Will vitamin therapy help?). Similarly, if the glaucoma classification module 412B determines that the patient likely has POAG (or another tested-for form of glaucoma), the classification modules specific to the glaucoma diagnosis can be executed (e.g., is the glaucoma severe?).

After the ensembles 412A-N process the OCT scan data, the host computer system 406 may then display the determinations on a screen (not shown) of the host computer system and/or transmit data indicative of the determination to another (e.g., remote) computer system 417. One such example is a computer system associated with the caregiver that performed the OCT scan and/or a computer system associated with the patient's health insurance provider.

In that connection, FIG. 1D is a flowchart of a process that the host computer system may implement according to various embodiments of the present invention. At step 500, the host computer system 406 receives the patient OCT scan image data. At step 501, the host computer system 406 pre-processes the patient OCT scan image data. More details about pre-processing are described below. Then, at steps 502A-C, the classification modules 412A-C can be executed to test for their respective tested-for conditions. The classification modules 412A-C can be executed in parallel as suggested by FIG. 1D, or they can be executed serially in various embodiments. Then, depending on whether the primary classification modules 412A-C test positive for their tested-for condition, the follow-up questions for the positive tested-for conditions can be executed at steps 504A-C. Some conditions may have more follow-up condition classifications or questions than others. And when the processing is complete at step 505, the results can be transmitted at step 506.

Preferably, the results of the host computing system 406 are provided shortly after the patient's OCT scan is taken, so that the caregiver can provide and review the results with the patient during the patient's appointment. For example, the OCT scanner 402 could be located in the office or facility of the patient's primary care provider. When the patient comes in for an appointment, the patient's retina can be scanned with the OCT scanner 402 and the results are sent to the host computer system 406. Within time of a normal office visit, e.g., 10 to 30 minutes, the host computer system 406 can transmit the results back to the remote computer system 417 at the primary care provider's facility/office, so that=the patient can get the results during his/her visit.

The classification modules 412A-N may each use an ensemble of traditional machine learning and/or deep learning techniques that are trained on training data to make their respective classifications. The machine learning techniques of the modules 412A-N can comprise, for example, both applied deep learning models and traditional machine learning (i.e., non-deep learning) models. The traditional machine learning models can comprise, for example, decision tree learning, shallow artificial neural networks, support vector machines, and rule-based machine learning. Deep learning on the other hand is machine learning based on learning data representations implicitly. Deep learning architectures may include several neural networks (e.g., deep, feed forward convolutional networks such as convolutional neural networks (CNNs)) and various recursive neural networks. Typically, neurons in a neural network are organized in layers. Different layers may perform different kinds of transformations on their inputs. Signals travel from the first (input), to the last (output) layer, possibly after traversing the layers multiple times. Networks with multiple hidden layers are deep neural networks (DNNs).

A neural network, shallow or deep, is a computing system that learns (progressively improves performance) to do tasks, such as to detect a certain disease or condition in OCT scan image data, by considering examples, generally without task-specific programming. An Artificial Neural Network (ANN) comprises a collection of connected units called artificial neurons. Each connection between neurons can transmit a signal to another neuron. The receiving neuron can process the signal(s) and then signal downstream neurons connected to it. Neurons may have a state, generally represented by real numbers, typically between 0.0 and 1.0. Neurons and connections may also have a weight that varies as learning proceeds, which can increase or decrease the strength of the signal that it sends downstream. Further, they may have a threshold such that the signal is sent downstream only if the aggregate signal is below (or above) that level is the downstream signal sent.

In the training process, the statistical models for the modules 412A-N are generated from a database or library of OCT scan image training data, where the test subjects whose scan data are composed of the database/library are classified as positive or negative for each classification question (e.g., whether they have ARMD or not, etc.). That is, for example, to generate a classification module 412A for ARMD, there should be sufficient and equally distributed amounts of training data in the database/library where the test subjects are known to both have wet ARMD, or have dry ARMD. From the positive and negative samples, the classification module 412A can train each of its one or more statistical models to classify, once trained, whether particular OCT scan data for patient should be classified as indicating that the patient has wet or dry ARMD (or more particularly, the classification module 412A can compute the likelihood that the patient has ARMD based on its statistical model(s)). Similarly, if an ARMD follow-up classification module 412N determines whether the patient would benefit from vitamin therapy (assuming the patient was determined to likely have ARMD by the first classification module 412A), the statistical model(s) of the follow-up classification module 412N can be trained on OCT scan data for ARMD-positive patients that both benefitted from vitamin therapy (positive samples) and did not benefit from vitamin therapy (negative samples). Still further, if another classification module 412N determines whether the ARMD-positive patient has wet ARMD, the statistical model(s) of that follow-up classification module 412N can be trained on OCT scan data for ARMD-positive patients that both have wet ARMD (positive samples), or have dry ARMD (negative samples). And so on for the other eye diseases, follow-up questions, and other classification modules, which can classify other relevant and applicable follow-up questions. Thus, the training data preferably has to be classified (Dry Macular Degeneration, Normal Eye, Wet Macular Degeneration without treatment, Treated Wet Macular Degeneration (needs injection)).

Preferably, each module 412A-N includes an ensemble of machine learning models, with the ensembles comprising both traditional machine learning models as well as deep learning models. Deep learning on large image datasets is an extremely effective technique for classification, but it may require large amounts of data to converge in order to obtain excellent performance. Another reason for its huge training data requirement is number of parameters to learn in the training phase. Increasing the current Deep Learning network by one layer of neurons leads to a huge amount of new parameters (weights) to be learned, which in turn require large amounts of data. Traditional machine learning models, such as decision tree, random forests, and support vector machines (SVMs), generally require less data to converge to optimal performance, but in some cases, may not achieve the same level of performance, and more importantly precision and recall as deep learning does. Accordingly, the classification modules 412A-N preferably includes multiple models from both traditional machine learning and deep learning paradigms, that leverage the relative strengths of each approach into a single ensemble that provides high accuracy as well as high generalizability. The ensemble may also be able to incorporate new image data and new rulesets to improve performance over the lifetime of the system.

The training data (see step 501 in FIG. 1D) is preferably pre-processed prior to training, of the classification modules 412A-N. The pre-processing stage can provide a number of benefits. First, it can reduce the noise in the data. Second, it can compress or reduce the dimensionality of the data so that the classification modules 412A-N can be trained or operate more efficiently. In that connection, among other things, the pre-processing can extract features from the OCT scan data that have been identified as potentially useful in making the desired classification. An incomplete list of methods that obtain these features includes:

Edge detection

Corner detection

Blob detection

Ridge detection

Scale-invariant transforms

Edge direction

Thresholding

Template matching

Hough transforms (Lines, Circles, etc.)

Active contours

Z-axis curve fitting

In most cases, it is also important to represent the training data in terms of disparate bases representations. This helps elicit feature components that encode specific characteristics of the representative image as well as the interaction between them. Some examples of the above mentioned bases representations include Principal Component Analysis (PCA), where the variance of the training data is captured; Karhunen-Loève Transform (KLT), which captures the energy of the training data; non-negative matrix factorization, which captures additive bases for the training data; Independent Component Analysis (ICA), which captures non-orthogonal variance of data; Gaussian basis representations of mixture models; or other forms of Eigen-based representations.

In various embodiments, the pre-processing primarily involves principal component analysis (PCA). PCA is a dimension-reduction algorithm/technique that can be used to reduce a large set of independent variables (e.g., in an OCT scan image) to a small set that still contains most of the information in the large set. In particular, it transforms a number of (possibly) correlated variables into a (smaller) number of uncorrelated variables called “principal components.” The first principal component accounts for as much of the variability in the data as possible, and each succeeding component accounts for as much of the remaining variability as possible. All the principal components are orthogonal/perpendicular to each other. Traditionally, principal component analysis is performed on a square symmetric matrix, such as a SSCP matrix, covariance matrix, or correlation matrix.

These feature extraction methods yield numeric data that create a data matrix for the image that can then be processed by, for example, a traditional machine learning method, such as a K-Nearest Neighbor (KNN) algorithm, a Decision Tree, or any other suitable traditional machine learning method. A traditional machine learning method can be extremely powerful if the underlying features are representative of the sources of variance in the underlying system. Ideal features should be system bases, or in other words, singular (no redundancy of information between features), and maximally informative (represent the complete variance in the measured dimension).

Deep Neural Networks, a type of applied deep learning method(s), differ from the traditional machine learning methods described above because they simultaneously transform, explore, and fit mathematical functions to all possible feature derivatives (e.g., using nonlinear transformations like a hyperbolic tangent or a rectilinear unit) from an original set of features. This makes Deep Neural Networks incredibly powerful non-linear learners. However, they may also require extremely large amounts of information to train effectively and may exhibit more undesirable behavior, such as overfitting or underfitting, than other machine learning methods. Overfitting occurs when the model learnt from the training data describes too well the underlying training system but not the unseen test data. On the other hand, underfitting happens when the model cannot learn from the training set. Both problems deter the model to generalize accordingly. Deep neural networks follow a defined architecture and model performance is very sensitive to network architecture, activation function selection, pooling layer size, and initialization settings. They can be trained, for example, with a backpropagation algorithm, which is a method to calculate the gradient of the loss function with respect to the weights for the nodes and connections in the network. Deep Neural Network performance may be difficult to replicate, even across the same data, unless identical settings and architectures are used. There are two currently common classes of deep neural network: 1) convolutional neural network (CNN) and 2) recursive neural network. Both classes of deep neural network may be used in this invention.

To take advantage of the power in Deep Neural Networks while mitigating their hazards, each of the classification modules 412A-N can include as part of their ensemble one or more deep neural network models combined with one or more traditional machine learning models. Embodiments of the present invention can you various classes of deep neural network particularly convolutional neural networks, recursive neural networks such as recurrent neural networks (RNN), recurrent convolutional neural networks (RCNN), long-short term memory (LSTM) and Capsule Nets among others. The dimensions of pooling layers, network initialization states, activation function, and trained network weightings may be unique to the applications for this invention.

The long-short term memory (LSTM) and recurrent convolutional neural networks (RCNN) are promising approaches particularly because of the relationships they capture through the hidden layers. Additionally, the objects detected within them can be considered as feature transformed bases on which a separate algorithm can make a prediction.

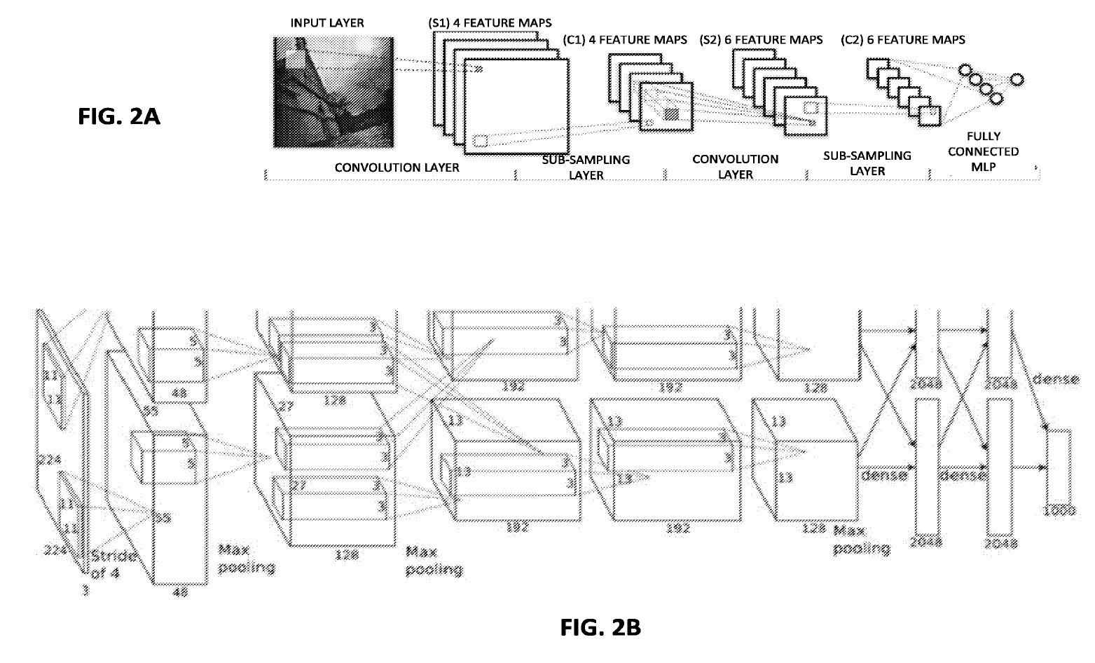

The Deep Neural Network model architectures may borrow components from popular architectures such as LeNet and/or AlexNet convolutional neural networks. FIG. 2A displays a graphical depiction of a LeNet model and FIG. 2B displays a graphical representation of AlexNet. LeNet and AlexNet are examples of convolutional deep neural networks that can provide high-performance image classification when correctly modified to fit the sizes of the images in the dataset and provided with adequately labeled data. Sparse, convolutional layers and max-pooling are at the heart of the LeNet family of models. As shown in FIG. 2A, lower-layers are composed to alternating convolution and max-pooling layers. The upper-layers however are fully-connected and correspond to a traditional multilayer perceptron (MLP) (hidden layer plus logistic regression). The input to the first fully-connected layer is the set of all features maps at the layer below. From an implementation point of view, this means lower-layers operate on 4D tensors. These are then flattened to a 2D matrix of rasterized feature maps, to be compatible with a MLP implementation. As shown in FIG. 2B, AlexNet has many layers. The first 5 are convolutional and the last 3 are fully connected layers. In between there can also be some pooling and activation layers. More details about LeNet and AlexNet are provided in “Deep Learning Tutorial,” Release 0.1, LISA Lab, University of Montreal, September 2015 (available at deeplearning.net/tutorial/lenet.html), Y. LeCun et al., “Gradient-Based Learning Applied to Document Recognition,” Proceedings of the IEEE, 86(11):2278-2324, November 1998, and Krizhevsky, A., et al., “ImageNet: Classification with Deep Convolutional Neural Networks,” NIPS 2012: Neural Information Processing Systems, Lake Tahoe, Nev. (2012), which are incorporated herein by reference in their entirety. As additional data is obtained, GoogLeNet may also be incorporated into the network architecture, as may other suitable neural networks. Furthermore, neural network architectures that perform more optimally on OCT scan data may also be combined with one or more of these architectures.

Depending on the nature of the additional data obtained, the models may be modified to leverage any time-series data available as part of a Recurrent Neural Network (RNN), which in practice works more optimally with information-rich sequential data. The decisions for the various dimensions may be dependent on empirical determination. More details that may drive dimension decisions are available in (1) Lipton Z C, Berkowitz J, Elkan C, “A critical review of recurrent neural networks for sequence learning,” arXiv:1506.00019 [cs.LG], 2015 and (2) A. Karpathy, J. Johnson, and L. Fei-Fei, “Visualizing and understanding recurrent networks,” arXiv:1506.02078, 2015, both of which are incorporated herein by reference in their entirety.

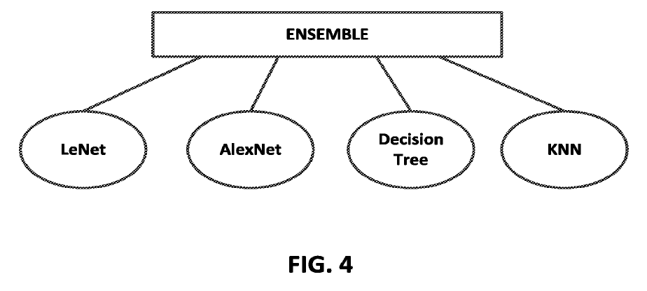

Alongside deep learning methodologies discussed above, there are more traditional strategies based on ensemble learning which can also be used. Boosted or Bootstrap Aggregation, also known as “bagging,” is a method of combining multiple sub-models (e.g., the deep learning and traditional machine learning algorithms in the ensemble) into a single model that retains an optimum level of performance. According to various embodiments, the sub-models in the ensemble may be any number of deep learning and/or traditional machine learning models as described above. In one embodiment, individual models may be trained on random subsets of data repeatedly, and the resulting models are combined into an ensemble, where the resulting models are combined using a simple linear function, such as the maximum votes among ensemble members. The various models may be trained based on the training OCT scan data, as described earlier. There is no inherent limitation to the type or number of models that can be combined in the classification modules 412A-N. As shown in the example of FIG. 4, a possible ensemble may include, for example, a LeNet architecture model, an AlexNet architecture model, a Decision Tree model, and a KNN model. In implementation, the classification modules 412A-N may have more or fewer deep learning and/or traditional machine learning models, and/or different kinds of deep learning and/or traditional machine learning models, such as RNNs, RCNNs, and LSTMs, as described above.

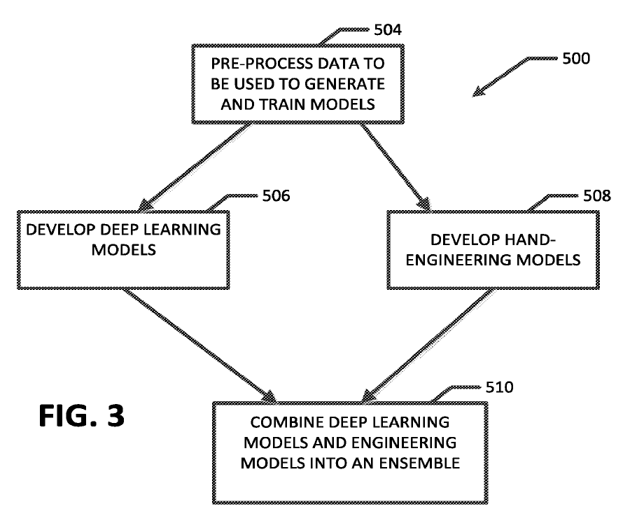

FIG. 3 displays an embodiment of the method 500 for developing and training the models that make up the ensemble of any one of the classification modules 412A-N. The method of FIG. 3 may be implemented with a suitably programmed computer system, such as the host computer system 406. First, at step 504, the training OCT scan data, which may be in a Digital Imaging and Communications in Medicine (DICOM) format, is pre-processed. The pre-processing can include PCA, ICA, transformation, normalization, mean subtraction, and/or whitening. Once the data have been preprocessed, the data may be then be used in training the deep learning models at step 506 and the traditional machine learning models at step 508.

The models can then be tested on the training data. If a model shows insufficient performance, it is not included in the ensemble. Conversely, if the model's performance is successful, it can be included in the ensemble. As an example, based on the performance obtained on the training set, the threshold will segregate the successful from the unsuccessful model to be aggregated in the ensemble.

The performance decision, in this context, can be based on the F-Measure and the Receiver Operating Characteristic (ROC) curve to determine the threshold that a model must exceed in order to be included in the ensemble. The F-Measure is a statistical analysis approach that considers precision and recall, which are fundamental in the medical context, and measures the effectiveness of the model. Additionally, the ROC curve measures the capability of the model to distinguish between two outcomes. ROC curve takes into consideration the sensitivity (true positive rate) as a function of the specificity (false positive rate).

This process can be performed for each model that is generated for the training data.

The models developed in steps 506 and 508 may then be combined at step 510 to form the ensemble for the particular classification module 412. The classification module 412 uses a decision criterion to combine the results from the various models in the ensemble, such as a predetermined weighting method. For example, each model could be weighted evenly with a majority rules criteria such that if a majority of the models in the ensemble classify the patient as having the condition, the decision of the ensemble is that the patient has the condition, and vice versa. Other weighting methods could also be used, such as to weight higher models that tend to be more accurate. Also, the classification module 412 can generate “soft” results, such probabilities that the patient has the tested-for condition, rather than a binary positive-negative Decision.

The models of the modules 412A-N may continue to be trained after going into testing stage.

In various other embodiments, the host computer system 406 may include classification modules tuned to other diseases that can be detected from OCT scan image data by such statistical models. For example, the classification modules could be trained to detect non-eye related diseases that are detectable through OCT retina scan image data, such as cardiovascular, Alzheimer's and/or Parkinson's disease. Again, such a classification module would need to be trained with a sufficient number of samples for that particular task/disease. And the classification module(s) would preferably include an ensemble of task-specific machine learning models and deep learning models, as described above.

Therefore, in one general aspect, the present invention is directed to an apparatus that comprises an OCT scanner 402 and a host computer system 406. The OCT scanner 402 captures patient scan image data of a retina of a patient, where the patient scan image data comprises 3-dimensional image data of the patient's retina. The host computer system 406 receives the patient scan image data of the patient's retina captured by the OCT scanner 402. The host computer system 406 comprises a plurality of classification modules 412A-N that make separate classifications based on the patient scan image data of the patient. The plurality of classification modules 412A-N are pre-trained on labeled OCT scan image training data that is pre-processed prior to training the classification modules, where the pre-processing comprises a principal component analysis (PCA) of the labeled OCT scan image training data. The plurality of classification modules 412A-N comprises: (i) a first classification module 412A that, when executed by the host computer system 406, determines a likelihood that the patient has ARMD; (ii) a second classification module 412B that, when executed by the host computer system 406, determines a likelihood that the patient has glaucoma; and (iii) a third classification module 412C that, when executed by the host computer system 406, determines a likelihood that the patient has diabetic retinopathy. Each of the first, second and third modules 412A-C comprises an ensemble of machine learning algorithms for making their classifications. In addition, the host computer system 406 transmits the determinations of the first, second and classification modules to a remote computer system 417, which may be co-located with the OCT scanner 402. For example, the OCT scanner 402 and the remote computer system 416 could be co-located at a primary care facility of the patient, and the host computer system 406 transmits the determinations of the first, second and classification modules 412A-C to the remote computer system 417 within 10 to 30 minutes of the OCT scanner 402 capturing the scan image data of the patient's retina.

In another general aspect, the present invention is directed to a method that comprises the step of pre-processing, by the host computer system 406, labeled OCT scan image training data, where the pre-processing comprises prior a principal component analysis (PCA) of the labeled OCT scan image training data. The method further comprises the steps of, after pre-processing the labeled OCT scan image training data, training, by the host computer system 406, a plurality of classification modules 412A-N of the host computer system 406, where the plurality of classification modules 412A-N are trained with the pre-processed labeled OCT scan image training data. The plurality of classification modules may comprise: (i) a first classification module 412A that, when executed by the host computer system 406, determines a likelihood that a patient has ARMD; (ii) a second classification module 412B that, when executed by the host computer system 406, determines a likelihood that the patient has glaucoma; and (iii) a third classification module 412C that, when executed by the host computer system 406, determines a likelihood that the patient has diabetic retinopathy. The method further comprises the step of capturing, by the OCT scanner 402, patient scan image data of a retina of a patient, where the patient scan image data comprises 3-dimensional image data of the patient's retina. The method further comprises the step of receiving, by the host computer system 406, the patient scan image data captured by the OCT scanner 402. The method further comprises the steps of: (i) determining, by the host computer system, by execution of the first classification module 412A, a likelihood that the patient has ARMD; (ii) determining, by the host computer system 406, by execution of the second classification module 412B, a likelihood that the patient has glaucoma; and (iii) determining, by the host computer system 406, by execution of the third classification module 412C, a likelihood that the patient has diabetic retinopathy. The method further comprises the step of transmitting, by the host computer system 406, the determinations of the first, second and classification modules to a remote computer system.

In various implementations, the ensembles for each of the first, second and third classification module 412A-C respectively comprises at least one deep learning algorithm and at least one traditional machine learning (i.e., non-deep learning) algorithm.

In various implementations, the host computer system 406 comprises a fourth classification module that determines, when executed by the host computer system 406, a feature of the patient's ARMD upon a determination by the first classification module 412A that the likelihood that the patient has ARMD is above a threshold level. The feature may be whether the patient has wet ARMD or whether the patient will benefit from vitamin therapy, for example. The fourth classification module may also comprise an ensemble of machine learning algorithms for making the classification, where the ensemble comprises at least one deep learning algorithm and at least one traditional machine learning algorithm. The host computer system 406 may also transmit the determination of fourth classification module to the remote computing system 417.

Similarly, the host computer system 406 may also include a classification module that determines, when executed by the host computer system, a feature of the patient's glaucoma upon a determination by the second classification module 412B that the likelihood that the patient has glaucoma is above a threshold level. That classification module may also comprise an ensemble of machine learning algorithms for making the classification, where the ensemble comprises at least one deep learning algorithm and at least one traditional machine learning algorithm. The host computer system 406 may also transmit the determination of the classification module to the remote computing system 417

Reference throughout the specification to “various embodiments,” “some embodiments,” “one embodiment,” “an embodiment”, “one aspect,” “an aspect” or the like, means that a particular feature, structure, or characteristic described in connection with the embodiment is included in at least one embodiment. Thus, appearances of the phrases “in various embodiments,” “in some embodiments,” “in one embodiment”, or “in an embodiment”, or the like, in places throughout the specification are not necessarily all referring to the same embodiment. Furthermore, the particular features, structures or characteristics may be combined in any suitable manner in one or more aspects. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation. Such modifications and variations are intended to be included within the scope of the present invention.

Although various embodiments have been described herein, many modifications, variations, substitutions, changes, and equivalents to those embodiments may be implemented and will occur to those skilled in the art. It is therefore to be understood that the foregoing description and the appended claims are intended to cover all such modifications and variations as falling within the scope of the disclosed embodiments. The following claims are intended to cover all such modification and variations.

In summary, numerous benefits have been described which result from employing the concepts described herein. The foregoing description of the one or more embodiments has been presented for purposes of illustration and description. It is not intended to be exhaustive or limiting to the precise form disclosed. Modifications or variations are possible in light of the above teachings. The one or more embodiments were chosen and described in order to illustrate principles and practical application to thereby enable one of ordinary skill in the art to utilize the various embodiments and with various modifications as are suited to the particular use contemplated.

What is claimed is:

1. An apparatus comprising:
an OCT scanner for capturing patient scan image data of a retina of a patient, wherein the patient scan image data comprises 3-dimensional image data of the patient's retina; and
a host computer system, wherein:
the host computer system receives the patient scan image data of the patient's retina captured by the OCT scanner;
the host computer system comprises a plurality of classification modules that make separate classifications based on the patient scan image data of the patient;
the plurality of classification modules are pre-trained on labeled OCT scan image training data that is pre-processed prior to training the classification modules, wherein the pre-processing comprises a principal component analysis (PCA) of the labeled OCT scan image training data;
the plurality of classification modules comprises:
a first classification module that, when executed by the host computer system, determines a likelihood that the patient has ARMD;
a second classification module that, when executed by the host computer system, determines a likelihood that the patient has glaucoma; and
a third classification module that, when executed by the host computer system, determines a likelihood that the patient has diabetic retinopathy;
each of the first, second and third modules comprises an ensemble of machine learning algorithms for making their classifications; and
the host computer system transmits the determinations of the first, second and classification modules to a remote computer system.

2. The apparatus of claim 1, wherein:
the ensemble for the first classification module comprises at least one deep learning algorithm and at least one traditional machine learning algorithm;
the ensemble for the second classification module comprises at least one deep learning algorithm and at least one traditional machine learning algorithm; and
the ensemble for the third classification module comprises at least one deep learning algorithm and at least one traditional machine learning algorithm.

3. The apparatus of claim 2, wherein the remote computer system is co-located with the OCT scanner.

4. The apparatus of claim 2, wherein the OCT scanner and remote computer system are co-located at a primary care facility of the patient, and the host computer system transmits the determinations of the first, second and classification modules to the remote computer system within 30 minutes of the OCT scanner capturing the scan image data of the patient's retina.

5. The apparatus of claim 4, wherein:
the host computer system comprises a fourth classification module that determines, when executed by the host computer system, a feature of the patient's ARMD upon a determination by the first classification module that the likelihood that the patient has ARMD is above a threshold level;
the fourth classification module comprises an ensemble of machine learning algorithms for making the classification;
the ensemble for the fourth classification module comprises at least one deep learning algorithm and at least one traditional machine learning algorithm; and
the host computer system transmits the determination of fourth classification module to the remote computing system.

6. The apparatus of claim 5, wherein the feature of the patient's ARMD classified by the fourth classification module is whether the patient has wet ARMD.

7. The apparatus of claim 5, wherein the feature of the patient's ARMD classified by the fourth classification module is whether the patient will benefit from vitamin therapy.

8. The apparatus of claim 4, wherein:
the host computer system comprises a fourth classification module that determines, when executed by the host computer system, upon a determination by the first classification module that the likelihood that the patient has ARMD is above a threshold level, whether the patient has wet ARMD;
the host computer system comprises a fifth classification module that determines, when executed by the host computer system, upon a determination by the first classification module that the likelihood that the patient has ARMD is above a threshold level, whether the patient will benefit from vitamin therapy;
the fourth and fifth classification modules each comprise an ensemble of machine learning algorithms for making their respective classifications;
the ensembles for the fourth and fifth classification modules each comprise at least one deep learning algorithm and at least one traditional machine learning algorithm; and
the host computer system transmits the determinations of fourth and fifth classification modules to the remote computing system.

9. The apparatus of claim 8, wherein
the host computer system comprises a sixth classification module that determines, when executed by the host computer system, a feature of the patient's glaucoma upon a determination by the second classification module that the likelihood that the patient has glaucoma is above a threshold level;
the six classification module comprises an ensemble of machine learning algorithms for making the classification;
the ensemble for the sixth classification module comprises at least one deep learning algorithm and at least one traditional machine learning algorithm; and
the host computer system transmits the determination of sixth classification module to the remote computing system.

10. The apparatus of claim 1, wherein
the first classification module combines the first ensemble of machine learning algorithms of the first classification module using a first bootstrap aggregation algorithm;
the second classification module combines the ensemble of machine learning algorithms of the second classification module using a second bootstrap aggregation algorithm; and
the third classification module combines the ensemble of machine learning algorithms of the third classification module using a third bootstrap aggregation algorithm

11. A method comprising:
pre-processing, by a host computer system, labeled OCT scan image training data, wherein the pre-processing comprises prior a principal component analysis (PCA) of the labeled OCT scan image training data;
after pre-processing the labeled OCT scan image training data, training, by the host computer system, a plurality of classification modules of the host computer system, wherein the plurality of classification modules are trained with the pre-processed labeled OCT scan image training data, and wherein the plurality of classification modules comprises:
a first classification module that, when executed by the host computer system, determines a likelihood that a patient has ARMD;
a second classification module that, when executed by the host computer system, determines a likelihood that the patient has glaucoma; and
a third classification module that, when executed by the host computer system, determines a likelihood that the patient has diabetic retinopathy;
capturing, by an OCT scanner, patient scan image data of a retina of a patient, wherein the patient scan image data comprises 3-dimensional image data of the patient's retina;
receiving, by the host computer system, the patient scan image data captured by the OCT scanner;
determining, by the host computer system, by execution of the first classification module, a likelihood that the patient has ARMD;
determining, by the host computer system, by execution of the second classification module, a likelihood that the patient has glaucoma;
determining, by the host computer system, by execution of the third classification module, a likelihood that the patient has diabetic retinopathy; and
transmitting, by the host computer system, the determinations of the first, second and classification modules to a remote computer system.

12. The method of claim 11, wherein:
the ensemble for the first classification module comprises at least one deep learning algorithm and at least one traditional machine learning algorithm;
the ensemble for the second classification module comprises at least one deep learning algorithm and at least one traditional machine learning algorithm; and
the ensemble for the third classification module comprises at least one deep learning algorithm and at least one traditional machine learning algorithm.

13. The method of claim 11, wherein:
the OCT scanner and remote computer system are co-located at a primary care facility of the patient; and
transmitting the determinations comprises transmitting by the host computer system transmits to the remote computer system within 30 minutes of the OCT scanner capturing the scan image data of the patient's retina.

14. The method of claim 12, wherein:
the host computer system comprises a fourth classification module that determines, when executed by the host computer system, a feature of the patient's ARMD upon a determination by the first classification module that the likelihood that the patient has ARMD is above a threshold level;
the fourth classification module comprises an ensemble of machine learning algorithms for making the classification;
the ensemble for the fourth classification module comprises at least one deep learning algorithm and at least one traditional machine learning algorithm; and
the host computer system transmits the determination of fourth classification module to the remote computing system.

15. The method of claim 14, wherein the feature of the patient's ARMD classified by the fourth classification module is whether the patient has wet ARMD.

16. The method of claim 14, wherein the feature of the patient's ARMD classified by the fourth classification module is whether the patient will benefit from vitamin therapy.

17. The method of claim 12, wherein:
the host computer system comprises a fourth classification module that determines, when executed by the host computer system, upon a determination by the first classification module that the likelihood that the patient has ARMD is above a threshold level, whether the patient has wet ARMD;
the host computer system comprises a fifth classification module that determines, when executed by the host computer system, upon a determination by the first classification module that the likelihood that the patient has ARMD is above a threshold level, whether the patient will benefit from vitamin therapy;
the fourth and fifth classification modules each comprise an ensemble of machine learning algorithms for making their respective classifications;
the ensembles for the fourth and fifth classification modules each comprise at least one deep learning algorithm and at least one traditional machine learning algorithm; and
the host computer system transmits the determinations of fourth and fifth classification modules to the remote computing system.

18. The method of claim 17, wherein
the host computer system comprises a sixth classification module that determines, when executed by the host computer system, a feature of the patient's glaucoma upon a determination by the second classification module that the likelihood that the patient has glaucoma is above a threshold level;
the six classification module comprises an ensemble of machine learning algorithms for making the classification;
the ensemble for the sixth classification module comprises at least one deep learning algorithm and at least one traditional machine learning algorithm; and
the host computer system transmits the determination of sixth classification module to the remote computing system.

19. The method of claim 11, wherein determining, by the host computer system, by execution of the first classification module, the likelihood that the patient has ARMD comprises combines the first ensemble of machine learning algorithms of the first classification module using a first bootstrap aggregation algorithm.
